(19) United States
(12) Patent Application Publication
(10) Pub. No.: US 2021/0388448 A1
(43) Pub. Date: Dec. 16, 2021

(54) METHOD OF DETERMINING PROGNOSIS IN PATIENTS WITH FOLLICULAR LYMPHOMA

(71) Applicants: MASARYKOVA UNIVERZITA, Brno (CZ); FAKULTNI NEMOCNICE BRNO, Brno (CZ)

(72) Inventors: Marek MRAZ, Brno (CZ); Jan DEVAN, Adamovske Kochanovce (SK); Katerina LITZMANOVIA, Brno (CZ)

(73) Assignees: MASARYKOVA UNIVERZITA, Brno (CZ); FAKULTNI NEMOCNICE BRNO, Brno (CZ)

(21) Appl. No.: 17/283,215

(22) PCT Filed: Oct. 14, 2019
(86) PCT No.: PCT/CZ2019/050047

§ 371 Date: Apr. 6, 2021

(30) Foreign Application Priority Data
Oct. 14, 2018 (CZ) PV 2018-546

(51) Int. Cl. C12Q 1/6886

(52) U.S. Cl. CPC C12Q 1/6886; C12Q 2600/166; C12Q 2600/178; C12Q 2600/118

(57) ABSTRACT

A method is disclosed for determining prognosis for a patient suffering from follicular lymphoma, that determines the amount of miR-31 in a biological sample taken from the body of the patient, and assigns the patient to a prognostic group based on the determined amount of miR-31, wherein the prognostic groups and the threshold values for assignment to the prognostic groups are obtained by analyzing the amount of miR-31 in biological samples of patients with known prognosis. In the biological sample taken from the patient, the miR-31 expression may be determined along with the expression of an endogenous control, which is a small nuclear RNA or stably expressed miRNA, and, in case of absolute quantification, the expression of synthetic standards of these miRNAs and endogenous controls of known number of molecules are determined. The method allows the assignment of patients to prognostic groups by determining miR-31 expression.

### FIELD OF ART

The present invention relates to a novel method of determining prognosis in patients suffering from follicular lymphoma.

### BACKGROUND ART

Follicular lymphoma (FL) is the most common type of indolent non-Hodgkin's lymphoma. Despite a significant improvement in the survival of patients after the introduction of immunotherapy, this disease, if diagnosed in advanced stages, still remains incurable (Benešová K. et al. Klin Onkol 2015; 28: 3S73-3S79). The course of FL is characterized by repeated relapses leading to the development of resistant disease. The fate of a particular patient with a newly diagnosed FL is still difficult to estimate. Several prognostic scores (FLIPI, FLIPI2) are being used, which are based mainly on basic clinical-laboratory parameters (age, clinical stage, laboratory activity of the disease usually expressed by means of levels of LDH, hemoglobin, beta-2-microglobulin etc.) or on clinical behavior over time (Casulo C. et al. J Clin Oncol Off J Am Soc Clin Oncol 2015; 33: 2516-2522). The main disadvantage of these scores is their low individual accuracy. Recently, a higher accuracy has been achieved by combining these prognostic scores with the mutation status of selected genes. These efforts have resulted in a new clinical-genetic prognostic score, in particular M7-FLIPI, which is based on the evaluation of the mutation status of 7 genes (EZH2, ARID1A, MEF2B, EP300, FOXO1, CREBBP and CARD11) in combination with clinical factors (FLIPI, ECOG scale) and modified M7-FLIPI designed to predict early disease progression, called POD24-PI (Jurinovic V. et al. Blood 2016; 128: 1112-1120; Pastore A. et al. Lancet Oncol 2015; 16: 1111-1122). The pathogenesis of FL results from a complex interaction of deregulated oncogenes, tumor suppressors, epigenetic regulators, and tumor microenvironment factors. Therefore, the prediction of patients' prognosis has hitherto mainly been based on a combination of many factors which should cover the considerable heterogeneity of the disease. Furthermore, determining new prognostic scores (M7-FLIPI, POD24-PI) requires sequencing of several genes in each patient and is therefore time-consuming and costly. In the case of POD24-PI, a long follow-up period of 24 months is also necessary. The aim of the present invention is thus to provide a novel method which enables rapid, accurate and cost-effective determination of the prognosis of FL patients.

### DISCLOSURE OF THE INVENTION

The present invention provides a method for determining a prognosis in a patient diagnosed with follicular lymphoma (FL), which comprises the step of determining the amount of miR-31 in a biological sample taken from a patient and the step of assigning the patient to a prognostic group based on the determined amount of miR-31, wherein the prognostic groups and threshold values for assignment to prognostic groups are obtained by analyzing the amount of miR-31 in biological samples of patients with known prognosis.

In general, a higher level of miR-31 is associated with a positive prognosis, i.e. a longer patients overall survival. Thus, within the scope of the present invention, a single marker has been identified which can largely replace the existing prognostic scores and the determination of which can be performed with high precision, using methods routinely available in diagnostic laboratories. A further advantage is also an easy availability of miRNA and thanks to its high stability also the possibility of isolation of miRNA from long-time archived FFPE blocks, frozen tissue, or the possibility of using circulating miRNA from body fluids.

The assigning of the patients into the prognostic groups is based on comparing the amount of miR-31 in the sample taken from the patient with a value corresponding to a selected quantile of the miR-31 amount in a group of patients with known prognosis. Patients with a miR-31 level lower than the said quantile are assigned to a prognostic group with a worse prognosis, and patients with a miR-31 level higher than the said quantile are assigned to a prognostic group with a better prognosis. For example, a prognosis may correspond to a prediction of survival for a predetermined period of time, for example, more than 3 years or more than 10 years, wherein the prognostic group with a worse prognosis has a low chance of survival (e.g. less than 70%, or 60% or less) during this period. The prognostic group with a better prognosis has a high chance of survival (for example at least 70%, or at least 80%, or at least 90%) during this period. For example, in one embodiment, the prognostic group with a worse prognosis has 55 to 60% survival at 10 years, while the prognostic group with a better prognosis has 90 to 92% survival at 10 years. In one embodiment, the quantile may be a 2nd tertile, but in other embodiments, the quantile may be chosen differently.

To use the amount of miR-31 as a prognostic marker, a reproducible quantification method was developed. Determination of the amount (expression) of miR-31 in a sample, i.e. quantification of miR-31, can be performed in a relative manner, wherein a normalized expression is expressed as the difference between the number of cycles needed to achieve a detection threshold for miR-31 and the number of cycles needed to achieve a detection threshold for an endogenous control; or in an absolute manner, wherein the number of miRNA molecules relative to 1,000,000 endogenous control molecules is calculated using synthetic standards. Relative quantification is procedurally simpler, but can only be used to compare the amounts of miR-31 determined in patients within a single laboratory or a single institution by the same method. To allow comparison of results and transfer of method between institutions or laboratories, it is preferable to perform the absolute quantification of miR-31, using a synthetic standard.

The quantification of miR-31 is performed relative to an endogenous control. The endogenous control is a substance whose amount (expression) in biological samples substantially does not change. In preferred embodiments of the invention, the endogenous control may be a small nuclear RNA (snoRNA), in particular selected from RNU38B, RNU6B, RNU44, RNU48, and/or the endogenous control may be a stably expressed miRNA, such as miR-16.

Thus, the expression of miR-31 is determined along with the expression of the endogenous control. In case absolute quantification is performed, the expression of the miR-31 synthetic standard and the endogenous control synthetic standard of known numbers of molecules is determined at the same time. Due to the nature of the miRNA, small nuclear RNA (snoRNA; e.g. RNU38B, RNU6B, RNU44, RNU48) and/or stably expressed miRNA (e.g. miR-16) can serve as the endogenous control. Within the framework of the present invention, synthetic standards have been designed (FIG. 1) for miR-31 and for the endogenous control, according to the evolutionarily conserved sequences of the given miRNAs and snoRNAs. These standards of known numbers of molecules are analyzed together with the samples, and then the exact copy numbers of miR-31 per million endogenous control molecules are calculated.

### Examples of the Designed Synthetic Standards

miR-31:
(SEQ ID NO. 1)
5′ - AGGCAAGAUGCUGGCAUAGCU - 3′
RNU38B:
(SEQ ID NO. 2)
5′ - CCA GUU CUG CUA CUG ACA GUA AGU GAA GAU AAA
GUG UGU CUG AGG AGA - 3′

Preferably, transcription of the miRNA into copyDNA (cDNA) and real-time quantitative polymerase chain reaction (qRT-PCR) detection are used to determine the expression. Using the determined miR-31 expression, the patients are divided into prognostic groups. MiR-31 expression thresholds are determined for individual prognostic groups. For relative quantification, normalized expression is expressed by the difference in the number of cycles required to achieve the detection threshold of miR-31 and the detection threshold of the endogenous control. For absolute quantification, the amount of the miRNA molecules relative to one endogenous control molecule is calculated directly, using synthetic standards. This procedure allows to reproduce the results between different laboratories.

Similarly, it is also possible to determine the expression of miR-31 using massive parallel sequencing/new generation sequencing techniques, i.e. to determine the number of miR-31 molecules relative to the number of endogenous control molecules.

In determining the prognosis, the expression of miR-31 in the sample taken from the patient is quantitatively determined and the patient is assigned to the corresponding prognostic group. The expression of miR-31 below the threshold value (given number of molecules in the sample) indicates a shortened survival time. The miR-31 threshold value (number of molecules in the sample) is obtained by statistical evaluation of the expression of miR-31 determined in biological samples taken from patients (miR-31 copies/million copies of endogenous control).

The determination of prognosis according to the invention relates solely to the progress of the follicular lymphoma disease; and not to the diagnosis of the transformation of follicular lymphoma (FL) into diffuse large B-cell lymphoma (DLBCL). Thus, the invention provides, by relative or absolute quantification of miR-31 in samples taken from a patient, a method of determining the prognosis of FL patients, without the need to rely on other known prognostic markers.

The biological sample may be a tumor tissue sample, for example, a frozen tissue, a sample of FFPE (formalin-fixed and paraffin-embedded sample) tumor tissue blocks, or a peripheral blood sample.

The quantification of miR-31, of the endogenous control, and optionally of the synthetic standard, is preferably performed by copyDNA (cDNA) and qRT-PCR detection, or the quantification is preferably performed using massive parallel sequencing/next generation sequencing (miRNAseq) techniques.

qRT-PCR is usually modified for use in the miRNA region, due to the specific properties of the miRNA. The biggest obstacle is the length of the miRNA itself, which corresponds to the length of commonly used primers, while shorter primers are not usable. In order to circumvent the problem of too low melting temperatures of the primers in duplex with miRNA, for example, commercially available stem-loop or hairpin primers can be used. The TaqMan® MicroRNA Assay (Life Technologies) detection system is based on the use of specially designed reverse transcription primers that extend miRNA, and the subsequent use of specific qRT-PCR probes. Reverse transcription is used to synthesize ss cDNA (ss=single stranded) from the isolated total RNA via the TaqMan® MicroRNA Reverse Transcription Kit (Life Technologies).

Primers and probes for the above methods for both miR-31 and endogenous controls are commercially available. For example, the TaqMan® MicroRNA Assay system (Life Technologies) was used in the examples below.

### BRIEF DESCRIPTION OF DRAWINGS

FIG. 1a. Range of Ct cycles (or copy numbers of miRNAs, respectively) covered by synthetic standards (Example 1).

FIG. 1b. Range of copies of molecules covered by synthetic standards (Example 1).

FIG. 2a. Patient survival as a function of miR-31 expression—dichotomization by second tercile (Example 2).

FIG. 2b. Patient survival as a function of miR-31 expression—dichotomization by second tercile (Example 2)

FIG. 2c. MiR-31 expression in patients who died within the first three years after biopsy versus expression in patients who survived past three years after biopsy.

### EXAMPLES OF CARRYING OUT THE INVENTION

### Example 1

Analysis of miR-31 expression in FL samples was performed to define the role of miR-31 in the FL prognosis. Both absolute and relative quantification of miR-31 were chosen as approaches. The relative quantification is technically simpler because it does not require the use of synthetic standards, but the results obtained can only be reliably applied within a single laboratory or institution. The absolute quantification, in contrast to relative quantification, allows an accurate determination of the number of miRNA copies in the sample, as well as offers the possibility to do this reproducibly in different laboratories. To calculate the copy number, a standard dilution series with known number of molecules was prepared to cover the required number of qRT-PCR cycles. Modification of qRT-PCR is required for its application in the miRNA region due to the specific properties of the miRNA. The biggest obstacle is the length of the miRNA, which corresponds to the length of commonly used primers, while shorter primers are not usable. To overcome the problem of the too low melting point of the primers in duplex with the miRNA, for example, commercially available stem-loop or hairpin primers were used. The TaqMan® MicroRNA Assay (Life Technologies) detection system is based on the use of specially designed reverse transcription primers that extend miRNA and subsequently of specific qRT-PCR probes. Reverse transcription is used to synthesize ss cDNA (ss=single strand) from the isolated total RNA using the TaqMan® MicroRNA Reverse Transcription Kit (Life Technologies). The starting RNA amount is approximately 4 ng of total RNA in a total reaction volume of 7.5 μl. Small nuclear RNA RNU38B was used as the endogenous control to normalize the miRNA expression; and miR-31 and RNU38B synthetic standards (according to miRBase v. 19; custom synthesis by Sigma Aldrich) were used to determine the absolute numbers of miR-31 molecules. Analogously, RNU6B, RNU44, RNU48 and other snoRNA can be used as control RNA.

miR-31:
(SEQ ID NO. 1)
5′ - AGGCAAGAUGCUGGCAUAGCU - 3′
RNU38B:
(SEQ ID NO. 2)
5′ - CCA GUU CUG CUA CUG ACA GUA AGU GAA GAU AAA
GUG UGU CUG AGG AGA - 3′

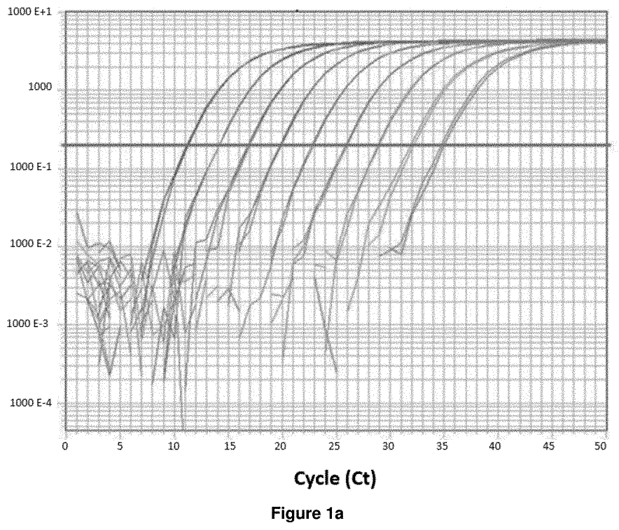

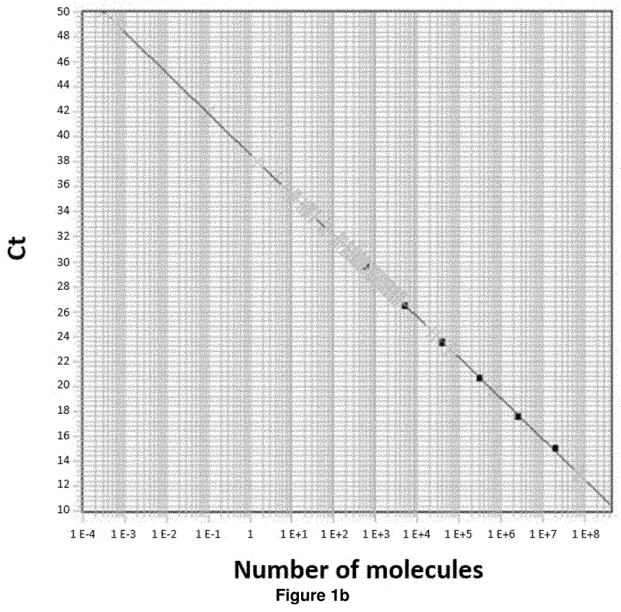

From these synthetically prepared RNAs, cDNA was prepared that was diluted 1:7 with nuclease-free water. The cDNA covers almost 20 cycles and a number of molecules in a reaction of approximately 70 to 18,106 molecules when amplified by qRT-PCR (FIG. 1a, b). The TaqMan® MicroRNA Reverse Transcription Kit (Life Technologies) was used to synthesize cDNA. Each sample was analyzed in triplicate using a TaqMan® Gene Expression Assay system (Life Technologies) according to the manufacturer's instructions. The cDNA amplification was then detected with the 7900 Real Time PCR System (Life Technologies). Data were evaluated in SDS v2.4 (Life Technologies) and then exported in Excel spreadsheet format (Microsoft Office XP). If the triplicate value deviation is greater than 0.3 cycles, the outlier value is discarded. Using absolute quantification, the number of miRNA molecules is converted to one million RNU38B endogenous control molecules. The use of the endogenous control has been validated along with the possible use of other small nuclear snoRNAs, eg RNU6B, RNU44, RNU48 and stably expressed miRNAs (miR-16). An analogous approach would involve determination of miR-31 expression by massive parallel sequencing/next generation sequencing techniques, i.e. to define the number of miR-31 molecules (see sequence above) relative to the number of endogenous control molecules (eg miR-16, see sequence above). Using relative quantification, normalized expression of a given miRNA is expressed only as a percentage of endogenous control expression, using the number of cycles required to reach the threshold fluorescence.

### Example 2

MiRNA from FFPE tissue blocks is isolated as recommended and using the chemicals of the High Pure miRNA Isolation Kit (Roche). To isolate RNA from paraffin, the tissue is first dewaxed and then digested with proteases. RNA in the presence of the chaotropic guanidine thiocyanate salt is bound to glass fibers in a column filter. Impurities are removed from the bound RNA by a series of washing steps and subsequently eluted with a salt-free aqueous solution (nuclease-free water). MiRNA is obtained by simply varying the concentration of column-binding-supporting buffer. Mixing a small amount of this buffer from the High Pure miRNA Isolation Kit (Roche) captures long RNA molecules while the miRNA passes through the column. Subsequently, the concentration of the buffer is increased and miRNA is captured in a new column.

RNA from frozen samples is isolated as recommended and using the TRIzol reagent (Life Technologies). The tissue sample is first homogenized in a 10-fold amount of TRIzol reagent and frozen at −70° C. After thawing, 1-bromo-3-chloro propane is added to allow separation of the upper clear phase containing RNA after centrifugation. The RNA-containing phase is collected and the RNA is precipitated with isopropanol, subsequently impurities were removed by a series of washing steps and dissolved in nuclease-free water.

The quality and concentration of the total RNA obtained is determined spectrophotometrically (NanoDrop) and electrophoretically (BioAnalyzer 2100). qRT-PCR is used to determine absolute gene expression. Samples of the tested miRNA and the endogenous control for each tested sample are always placed on one plate together with a series of standards to determine the absolute number of miR-31 and RNU38B molecules, or without standards for relative quantification. cDNA is applied in triplicate. To exclude contamination, duplicates are included in which the cDNA is replaced with nuclease-free water. An internal standard, which is a patient RNA sample (i.e. the same RNA throughout the assay series, detection of the same endogenous control and miRNA), is used to compare the plates with each other. Data are evaluated in SDS v2.4 and then exported in Excel spreadsheet format (Microsoft Office XP). If the triplicate value variation was greater than 0.3 cycles, the outlier value was discarded. In the case of absolute quantification, the number of miRNA molecules per million of RNU38B endogenous control molecules is subsequently calculated.

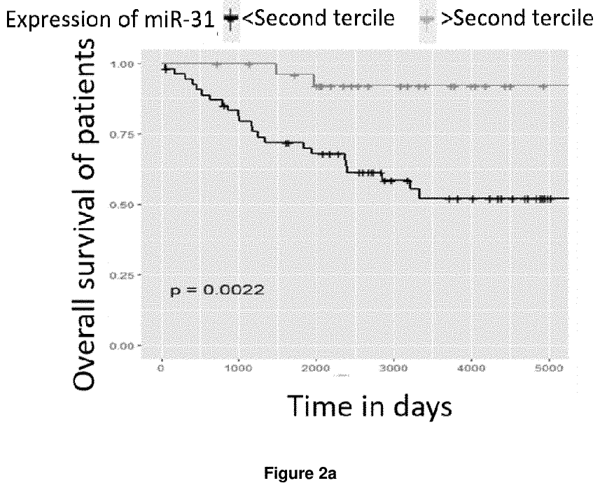

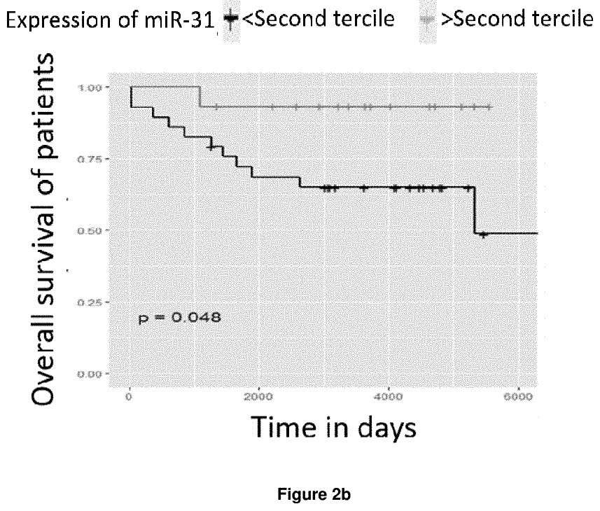

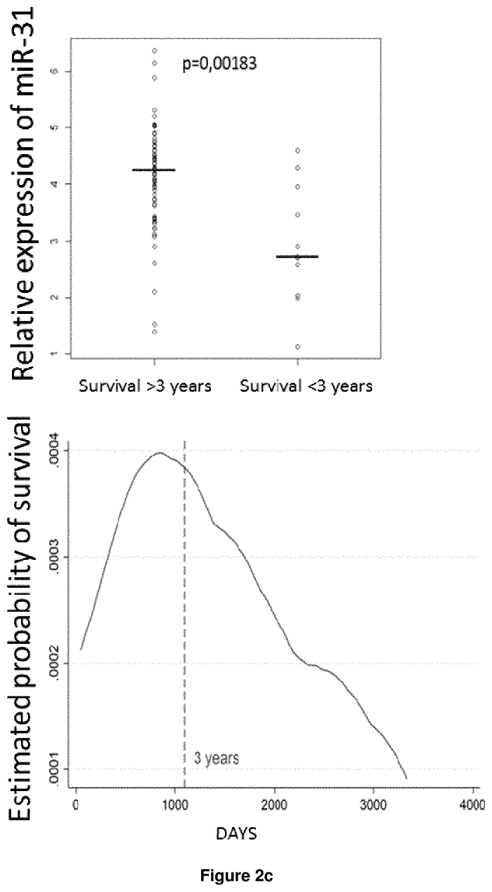

Using the miR-31 quantification procedure in FL tumor samples, FL patients can be divided into prognostic groups. In two independent cohorts of FL patients, it was shown that patients with miR-31 levels lower than 2nd tercile expression had significantly worse prognosis than patients whose miR-31 levels were higher than 2nd tercile expression in the cohort (first cohort: 55% vs. 90% surviving after 10 years; p<0.01; second cohort: 60% vs. 92% surviving after 10 years) (FIG. 2a, 2b). When using the second tercile as a threshold value, miR-31 is also a highly significant independent predictor of overall survival in a multivariate regression analysis that included several known prognostic markers (age, gender, hemoglobin, FLIPI score, LDH, presence of B-symptoms), affection of more than 4 lymph nodes, disease stage and miR-31 level (p=0.035; HR=8.90; Table 1). MiR-31 expression was also significantly lower in patients who died within the first three years after biopsy (FIG. 2c).

TABLE 1
Univariate and multivariate Cox regression analysis
for FL patient overall survival (n = 83)

| | | | |
|---|---|---|---|
| | Risk | 95% CI | 95% CI |
| P-value | ratio | (lower) | (upper) |

Univariate Cox regression of proportional risks

| | | | | |
|---|---|---|---|---|
| Gender (male) | 0.910 | 1.047 | 0.477 | 2.294 |
| Involvement of >4 nodes | 0.006 | 3.341 | 1.415 | 7.888 |
| Clinical stage III-IV | 0.058 | 2.826 | 0.964 | 8.281 |
| Presence of B symptoms | 0.443 | 1.394 | 0.596 | 3.258 |
| Increased LDH | 0.041 | 3.075 | 1.045 | 9.045 |
| Haemoglobin <120 g/l | 0.115 | 2.061 | 0.838 | 5.071 |
| Age >60 years | 0.105 | 1.931 | 0.871 | 4.283 |
| High score FLIPI (3-5) | 0.001 | 6.594 | 2.219 | 19.592 |
| miR-31 level <2. tercile | 0.009 | 6.944 | 1.637 | 29.463 |

Multivariate Cox regression of proportional risks

| | | | | | |
|---|---|---|---|---|---|
| Step 1 | Gender (male) | 0.725 | 0.843 | 0.327 | 2.177 |
| | Increased LDH | 0.379 | 0.465 | 0.085 | 2.561 |
| | Involvement of >4 nodes | 0.515 | 1.435 | 0.484 | 4.253 |
| | Clinical stage III-IV | 0.120 | 0.235 | 0.038 | 1.459 |
| | Presence of B symptoms | 0.411 | 0.656 | 0.241 | 1.790 |
| | Haemoglobin <120 g/l | 0.702 | 0.804 | 0.263 | 2.455 |
| | Age >60 years | 0.951 | 0.969 | 0.355 | 2.649 |
| | High score FLIPI (3-5) | 0.035 | 10.215 | 1.181 | 88.350 |
| | miR-31 level <2. tercile | 0.014 | 14.331 | 1.731 | 118.642 |
| Step 2 | Gender (male) | 0.697 | 0.836 | 0.338 | 2.064 |
| | Increased LDH | 0.381 | 0.468 | 0.085 | 2.560 |
| | Involvement of >4 nodes | 0.489 | 1.447 | 0.507 | 4.128 |
| | Clinical stage III-IV | 0.108 | 0.239 | 0.042 | 1.367 |
| | Presence of B symptoms | 0.409 | 0.655 | 0.241 | 1.786 |
| | Haemoglobin <120 g/l | 0.704 | 0.807 | 0.266 | 2.443 |
| | High score FLIPI (3-5) | 0.030 | 10.044 | 1.246 | 80.985 |
| | miR-31 level <2. tercile | 0.014 | 14.337 | 1.731 | 118.729 |
| Step 3 | Gender (male) | 0.713 | 0.844 | 0.342 | 2.083 |
| | Increased LDH | 0.422 | 0.514 | 0.102 | 2.602 |
| | Involvement of >4 nodes | 0.430 | 1.511 | 0.543 | 4.207 |
| | Clinical stage III-IV | 0.114 | 0.261 | 0.049 | 1.382 |
| | Presence of B symptoms | 0.351 | 0.628 | 0.236 | 1.670 |
| | High score FLIPI (3-5) | 0.026 | 8.456 | 1.285 | 55.630 |
| | miR-31 level <2. tercile | 0.014 | 14.031 | 1.716 | 114.694 |
| Step 4 | Increased LDH | 0.374 | 0.484 | 0.098 | 2.396 |
| | Involvement of > 4 nodes | 0.443 | 1.489 | 0.538 | 4.121 |
| | Clinical stage III-IV | 0.096 | 0.246 | 0.047 | 1.283 |
| | Presence of B symptoms | 0.357 | 0.633 | 0.239 | 1.676 |
| | High score FLIPI (3-5) | 0.020 | 9.083 | 1.418 | 58.166 |
| | miR-31 level <2. tercile | 0.014 | 13.818 | 1.693 | 112.821 |
| Step 5 | Increased LDH | 0.338 | 0.457 | 0.092 | 2.269 |
| | Clinical stage III-IV | 0.125 | 0.303 | 0.066 | 1.395 |
| | Presence of B symptoms | 0.416 | 0.671 | 0.257 | 1.756 |
| | High score FLIPI (3-5) | 0.013 | 10.204 | 1.647 | 63.244 |
| | miR-31 level <2. tercile | 0.017 | 12.972 | 1.589 | 105.924 |
| Step 6 | Increased LDH | 0.280 | 0.449 | 0.105 | 1.918 |
| | Clinical stage III-IV | 0.188 | 0.343 | 0.070 | 1.688 |
| | High score FLIPI (3-5) | 0.008 | 10.463 | 1.836 | 59.639 |
| | miR-31 level <2. tercile | 0.020 | 11.861 | 1.469 | 95.784 |
| Step 7 | Clinical stage III-IV | 0.324 | 0.455 | 0.095 | 2.176 |
| | High score FLIPI (3-5) | 0.011 | 6.088 | 1.510 | 24.543 |
| | miR-31 level <2. tercile | 0.028 | 10.166 | 1.292 | 79.956 |
| Step 8 | High score FLIPI (3-5) | 0.010 | 4.271 | 1.417 | 12.872 |
| | miR-31 level <2. tercile | 0.035 | 8.904 | 1.166 | 67.977 |

The left-truncated Cox regression model of proportional risks with delayed entry variables was used for overall survival as the biopsy time from diagnosis varied between patients. miR-31 was divided into two groups based on the 2nd tercile (equal to value 53) of its relative expression (normalized to RNU38B). CI—confidence interval.

1. A method for determining prognosis for a patient suffering from follicular lymphoma, comprising the steps of:
determining the amount of miR-31 in a biological sample taken from the body of the patient,
assigning the patient to a prognostic group based on the determined amount of miR-31,
wherein the prognostic groups and the threshold values for assignment to the prognostic groups are obtained by analyzing the amount of miR-31 in biological samples of patients with known prognosis.

2. The method of claim 1, wherein the threshold value is a quantile of miR-31 obtained by statistically evaluating the amount of miR-31 in biological samples of patients with known prognosis, wherein patients with the miR-31 level lower than the said quantile of the amount of miR-31 are assigned to a prognostic group with a shorter overall survival time, and patients with the miR-31 level lower than the said quantile of the amount of miR-31 are assigned to a prognostic group with a longer overall survival time.

3. The method according to claim 1, wherein the amount of miR-31 is determined relative to an endogenous control, preferably the endogenous control is selected from small nuclear RNA and stably expressed miRNA, more preferably the endogenous control is selected from RNU38B, RNU6B, RNU44, RNU48, and miR-16.

4. The method according to claim 1, wherein the amount of miR-31 is determined by absolute quantification using synthetic standards.

5. The method according to claim 1, wherein the amount of miR-31 and optionally of the endogenous control and/or of the synthetic standards is determined by copyDNA transcription and detection by qRT-PCR, or is determined by massive parallel sequencing/new generation sequencing techniques.

6. The method according to claim 1, wherein the biological sample taken from the body of the patient is selected from the group consisting of a tumor tissue sample such as frozen tumor tissue sample, a sample of FFPE blocks of tumor tissue, or a peripheral blood sample.

7. The method according to claim 4, wherein the synthetic standards are:
miR-31:
(SEQ ID NO. 1)
5′ - AGGCAAGAUGCUGGCAUAGCU - 3′
RNU38B:
(SEQ ID NO. 2)
5′ - CCA GUU CUG CUA CUG ACA GUA AGU GAA GAU AAA
GUG UGU CUG AGG AGA - 3′.


SEQUENCE LISTING (2 sequences)

SEQ ID NO: 1 - DNA, 21 bp, Artificial
  Synthetic standard, miR-31
aggcaagaugcuggcauagcu

SEQ ID NO: 2 - DNA, 48 bp, Artificial
  Synthetic standard RNU38B
ccaguucugcuacugacaguaagugaagauaaagugugucugaggaga